(19) United States
(12) Patent Application Publication
(10) Pub. No.: US 2003/0170332 A1
(43) Pub. Date: Sep. 11, 2003

(54) Flavonoid extract

(75) Inventors: Somasundaram Tharmalingam Senthilmohan, Christchurch (NZ); Roger Anthony Stanley, Auckland (NZ); Larry Ellsworth Stenswick, Rangiora (NZ)

Correspondence Address: GREER, BURNS & CRAIN, 300 S WACKER DR, 25TH FLOOR, CHICAGO, IL, 60606, US

(73) Assignee: Enzo Nutraceuticals Limited.

(21) Appl. No.: 10/327,769

(22) Filed: Dec. 23, 2002

(30) Foreign Application Priority Data
Dec. 24, 2001 (NZ) NZ 516 367

(51) Int. Cl. A61K 35/78

(52) U.S. Cl. 424/770; 424/775

(57) ABSTRACT

The use of a flavonoid extract is described to treat a mammal for at least one of the treatments including: reducing a redox imbalance associated with disease in a mammal; preventing oxidative stress in a mammal; reducing oxidative stress in a mammal; preventing protein oxidation in a mammal; reducing protein oxidation in a mammal; preventing DNA damage in a mammal; reducing DNA damage in a mammal; and combinations thereof. A method of treatment of a mammal is also described. The treatments have the advantage of maintaining health in an individual.

### BACKGROUND

The present invention relates to the use of a flavonoid extract for reducing or preventing protein damage and DNA damage in mammals. Preferably the present invention relates to the use of a proanthocyanidin-rich flavonoid extract from the bark of the species Pinus for reducing or preventing protein damage, more specifically protein oxidation and DNA damage in mammals.

The potential use of dietary supplements for protection against the effects of oxidative stress and the progression of degenerative diseases and aging has been the subject of an increasing number of studies during the past two decades. The effectiveness of antioxidants in vivo can be studied by their influence in reducing the level of bio-markers associated with oxidative damage.

Free radicals, reactive oxygen species (ROS) and reactive nitrogen species (RNS) are generated as by-products of normal cellular metabolism (Griffiths et al, 1998; Pryor & Squadrito, 1995). Their deleterious effects are minimized in-vivo by the presence of antioxidant systems, both enzymatic and scavenging (Griffiths et al, 1998; Frei et al, 1992). However if these damaging species are ineffectively scavenged, they can interact with biological macromolecules, such as DNA, lipids and proteins, with potential threat to cellular function. According to the free radical theory of aging, loss of cellular function during aging is a consequence of accumulating sub-cellular damage inflicted by activated oxygen species (Harman, 1981).

Bio-markers of oxidative damage to blood, such as oxidized lipids and proteins as well as damaged lymphocyte DNA, are thought to be useful indicators of oxidative stress that can be measured to indicate bio-availability and efficacy of antioxidant supplements. Protein carbonyl content is the most widely used marker of protein oxidation. Protein carbonyls are formed by a variety of oxidative mechanisms and are sensitive indices of oxidative injury. They appear to be one of the first changes seen with low amounts of oxidant (Ciolino & Levine, 1997). Furthermore the most prominent cell targets for the hydroxyl (HO.) free radicals are proteins (Lodish et al, 1995). The comet assay measures DNA strand breaks at the level of single cells, is very easily applied to lymphocytes and therefore lends itself to human bio-monitoring studies (Collins et al, 1997). It has become a standard technology for the measurement of oxidative DNA damage both in-vitro and in-vivo (Duthie et al, 1997; Beatty et al, 2001).

The antioxidant hypothesis postulates that supplementation with dietary antioxidants can alleviate the redox imbalance associated with disease. Many studies have demonstrated the protective properties of the polyphenolic flavonoids. Antimutagenic, anticarcihogenic and immune stimulating properties of flavonoids have been reported (Middleton, 1996; Middleton et. al, 1993). The flavonoids are a large group of naturally occurring polyphenols found in fruits, vegetables, grains, bark, tea and wine that have proven in-vitro free-radical scavenging potential (Johnson and Loo, 2000, Duthie et al, 1997a; Duthie et al, 1997b).

Pine bark extract is a commercially available proanthocyanidin-rich, flavonoid extract. One type is derived from the bark of Pinus radiata. When tested in basic solution, the superoxide scavenging ability of Pinus radiata bark extract was 13 times more effective as an antioxidant than vitamin C. Furthermore, in aqueous and acidic solutions, Pinus radiata bark extract acted as a more potent antioxidant than vitamin C, catechin, other bark extract, grape seed extracts and grape skin extracts (Wood et al, 2001). Phenolic compounds of Pinus radiata bark include catechin, epicatechin, quercetin, dihydroquercetin, taxifolin, phenolic acids, and procyanidin dimers, trimers, oligomers and polymers formed from catechin and epicatechin (Porter, 1974; Markham & Porter, 1973; Packer, Rimbach & Virgili, 1999).

No prior art however appears to establish the ability of any flavonoid or anti-oxidant extract to reduce both protein oxidation and DNA damage.

It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art in any country.

It is acknowledged that the term ‘comprise’ may, under varying jurisdictions, be attributed with either an exclusive or an inclusive meaning. For the purpose of this specification, and unless otherwise noted, the term ‘comprise‘ shall have an inclusive meaning—i.e. that it will be taken to mean an inclusion of not only the listed components it directly references but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### SUMMARY

According to one aspect of the present invention there is provided the use of a flavonoid extract in a treatment of a mammal for at least one of the treatments selected from the group including: to reduce a redox imbalance associated with disease in a mammal; to reduce oxidative stress in a mammal; to prevent oxidative stress in a mammal; to reduce protein oxidation in a mammal; to prevent protein oxidation in a mammal; to reduce DNA damage in a mammal; to prevent DNA damage in a mammal; and combinations thereof.

According to a further aspect of the present invention there is provided a method of treatment of a mammal including administration of a flavonoid extract in a combination treatment including at least one of the treatments selected from the group including: to reduce a redox imbalance associated with disease in a mammal; to reduce oxidative stress in a mammal; to prevent oxidative stress in a mammal; to reduce protein oxidation in a mammal; to prevent protein oxidation in a mammal; to reduce DNA damage in a mammal; to prevent DNA damage in a mammal; and combinations thereof.

According to a further aspect of the present invention there is provided the use of a flavonoid extract as described above wherein the extract is an anti-oxidant extract.

According to a further aspect of the present invention there is provided the use of a flavonoid extract as described above wherein said extract is a proanthocyanidin-rich extract. Preferably, the proanthocyanidin-rich extract is a pine bark extract. Most preferably the pine bark extract is from the species Pinus radiata .

According to a further aspect of the present invention there is provided the use of a flavonoid extract as described above wherein the extract is included in a dietary supplement formulation. Optionally, said formulation includes at least one additional anti-oxidant. Preferably, said anti-oxidant is vitamin C, and the ratio of extract to vitamin C is 2:1 by weight.

According to a further aspect of the present invention there is provided the use of a flavonoid extract as described above wherein the mammal is a human being.

According to a further aspect of the present invention there is provided the use of a flavonoid extract as described above wherein the daily dosage of said extract for a human is between 5 mg per day and 1500 mg per day, and is preferably 480 mg per day.

In a further preferred embodiment, the extract is taken orally. The extract may also be taken in any other known method of oral or other application, wherein the method of delivery is selected from: a tablet; a capsule; a suppository; an injection; a suspension; a drink; a tonic; a syrup; a powder; an ingredient in solid foods; an ingredient in liquid foods; a topical application; and combinations thereof.

The invention establishes that supplementation with dietary antioxidants can alleviate the redox imbalance associated with disease. The free radical scavenging ability of flavonoids can protect the human body from oxidative damage, which may cause many diseases including cancer, coronary heart disease and lead to the aging process (Middleton et al, 1993; Winterbourn, 1995). Studies have shown that increasing levels of flavonoids in the diet could decrease cancer and heart disease (Block, Patterson, & Subar, 1992; Hertog, Feskens, Hollman, Katan, & Kromhout, 1993). The consumption of flavonoids is beneficial for people of all ages, however seniors are at greater risk for protein and DNA damage than younger people. Ames et al reported that the level of metabolism is seven times higher in elderly rats than in adolescents, resulting in twice the number of DNA lesions (Ames, 1992).

Various authors have reported that the activity of the antioxidant enzymes such as superoxide dismutase (Niwa, 1990) and glutathione peroxidase (Santa Maria, 1987) do not change with the aging process. Further no correlation has been observed between catalase and glutathione peroxidase activities and the maximum life span of various species in some aging models. These results indicate that aging is not associated with a shortage of antioxidant enzyme protection in these models.

Flavonoids have been shown to possess a number of desirable biological effects, countering inflammatory, bacterial, viral, fungal, hormonal, carcinogenic, neoplastic and allergic disorders in both in-vitro and in-vivo systems (Middleton, 1996). The invention establishes that an increased intake of anti-oxidants aids in the prevention of some age related diseases.

The formation of carbonyl groups on amino acid residues as a result of free radical-initiated reactions is well-documented (Stadtman, 1990). Protein carbonyls are formed by a variety of oxidative mechanisms and are sensitive indices of oxidative injury (Stadtman, 1990). The formation of carbonyl groups occurs during normal aging (Carney 1991, Oliver 1987, Stadtman 1992 & Sohal 1994). Even in native plasma, there is a trend towards higher carbonyl levels in smokers than in controls (Marangon et al, 1999). Carbonyl formation is increased by oxidative stress (Reznick, 1994) and is associated with several diseases of humans including Alzheimer's disease, rheumatoid arthritis, and inflammatory bowel disease.

### BRIEF DESCRIPTION OF THE DRAWINGS

Further aspects of the present invention will become apparent from the following description, which is given by way of example only and with reference to the accompanying drawings in which:

FIG. 1 shows the effects on plasma protein carbonyls in elderly subjects, of flavonoid supplementation administered in accordance with the present invention; and

FIG. 2 shows the effects on plasma protein carbonyls between the genders of the subjects of flavonoid supplementation administered in accordance with the present invention.,

### DETAILED DESCRIPTION

The invention will now be further described with reference to more detailed examples.

Materials and Methods

Subjects

Twenty-six older human subjects aged between 55-75 years with the mean age 64, were recruited in a 12 week study. The subjects were recruited using an advertisement in a local newspaper. Selected subjects. were non-smokers without any significant clinical disease entities and were not taking any other medications or food supplements. They were free from diabetes mellitus, treated hypertension, hormone replacement therapy, malignancy, and any serious concomitant disorder. A brief medical history was obtained from each participant and included age, ethnicity, past and current medical disorders and smoking history. Two individuals were withdrawn from the study; one for an unrelated medical condition, the other for non-compliance. Twenty-four of the 26 subjects completed the study. The study was approved by the Ethics Committee, Christchurch Hospital, Christchurch, New Zealand.

Supplements

Capsules were supplied by ENZO Neutraceuticals Ltd (Christchurch, New Zealand) and consisted of Pinus radiata bark extract, (120 mg) and vitamin C (60 mg). The Pinus radiata bark extract was manufactured in accordance with NZ329658/ U.S. Pat. No.5,968,517 which is incorporated herein by reference.

The administration of the above-identified capsules embodied the present method, i.e., the use of a flavonoid extract wherein the extract is a proanthocyanidin-rich extract. Preferably, the proanthocyanidin-rich extract is a pine bark extract. Most preferably the pine bark extract is from the species Pinus radiata . The extract is included in a dietary supplement formulation. Optionally, the formulation includes at least one additional anti-oxidant. Preferably, said anti-oxidant is vitamin C, and the ratio of extract to vitamin C is 2:1 by weight.

The daily dosage of the extract for a human is between 5 mg per day and 1500 mg per day, and is preferably 480 mg per day. In a preferred embodiment, the extract is taken orally, but it is also contemplated that the extract may be taken in any other known method of oral or other application, wherein the method of delivery is selected from: a tablet; a capsule; a suppository; an injection; a suspension; a drink; a tonic; a syrup; a powder; an ingredient in solid foods; an ingredient in liquid foods; a topical application; and combinations thereof.

Subjects were instructed to consume 2 capsules prior to breakfast and 2 capsules prior to evening meal with a glass of water, providing 480 mg of Pinus radiata bark extract per day. The compliance of the study subjects was checked by carrying out a count of capsules returned at 6 and 12 weeks.

Blood Sampling

Fasting (12 hours) venous blood samples were collected into heparinised tubes for the carbonyl assay at baseline, after 6 and 12 weeks of supplementation. Samples were immediately placed under refrigeration, and plasma was separated by centrifugation. Plasma samples were stored at −80° C. until analyzed. Each sample was analyzed for protein carbonyls in triplicate. Measurement of DNA oxidative damage was carried out on isolated peripheral lymphocytes at baseline 6 weeks and 12 weeks. Isolation of peripheral lymphocytes was carried out based on procedure published by Smith et al (Smith 1999). Blood samples were collected into heparinized tubes. Peripheral lymphocytes were isolated from whole blood using density gradient centrifugation on Ficoll-Paque (Pharmacia Biotech A B, Uppsala, Sweden) at 1900×g for 20 minutes. Cells were collected, washed twice with sterile PBS, and frozen in 8% DMSO at −80° C. until analysed. Whole blood samples were frozen at −80° C. until analyzed.

Protein Carbonyl Assay

Plasma samples were analyzed for protein carbonyl concentrations as a measure of protein oxidation by an enzyme-linked immunosorbent assay (ELISA) method of Buss 1997. This was carried out using a Protein Carbonyl Enzyme Immuno-Assay Kit (Protein Carbonyl Kit, Zentec, Dunedin, NZ), which performs the measurement of protein carbonyls in biological samples. Protein carbonyls were reacted with 2,4-dinitrophenyl hydrazine (DNPH) and then the protein was non-specifically adsorbed onto an ELISA plate. The hydrazone adducts were detected with anti-DNP-biotin-antibody labeled with streptavidin-biotinylated horseradish peroxidase and reacted with chromatin. The absorbency was read at 450 nm directly after stopping reaction. Each sample was analyzed in triplicate and samples were quantitated by comparison with oxidized BSA standards (Buss 1997).

The alkaline comet assay was performed as described by Singh et al (1988) with modifications previously described by Tice (1998). Conventional frosted microscopic slides were dipped into hot 1.0% normal melting point agarose to one-half the frosted area and the underside of slide wiped to remove agarose. A 75 μl drop of 0.5% low melting point agarose (LMPA) at 37° C. was mixed with ˜10,000 cells in ˜5-10 μL of Ficol extract, and a coverslip was applied to spread the samples. After hardening the coverslip was removed and a third agarose layer (75 μl LMPA) was added, the coverslip reapplied and removed after the agarose layer hardened. The slides were lysed for 1 hour at 4° C. They were then soaked in a couplin jar containing electrophoresis solution to unwind for 40 minutes and electrophoresed at a constant current of 300 mA, for 35 minutes. After electrophoresis, the slides were neutralized with Tris-HCI buffer at pH 7.5 by three washes for 5 min each followed cold ethanol for 5 to 10 min and left to dry overnight. The slides were stained by placing 300 μl ethidium bromide solution (6 μg/mL) on each slide and covered with a coverslip for 20 minutes. They were then destained for 10 minutes in deionised water and viewed under an epifluorescence microscope (Zeiss epifluoresent) with an attached CCD camera and computer. Images were saved as electronic files and the Comets measured for comet tail length and tail moment based on the definition by Olive and Banath (1993). For each sample, 100 isolated comets were randomly selected and tail moments were measured.

Total Antioxidant Capacity

The total antioxidant capacity of Pinus radiata was assessed by using Oxygen Radical Absorbance Capacity (ORAC) assay. ORAC assay were carried out following procedures from a method previously described by Cao et al. (1993) using a PERKIN ELMER LS50B Luminescence Spectrometer equipped with the four-position, motor driven, water thermo stated, stirred cell holder. The final results (ORAC value) are expressed using trolox equivalents antioxidant capacity (TEAC value) based on the area under phycoerythrin decay curve.

Statistical Analysis

Statistical significance were determined by using paired and unpaired t-test (SigmaStat software) for the data. Results were considered as significant when the calculated p value was less than 0.05.

Results

The clinical trial was satisfactorily completed by twenty four (14 males & 10 females) subjects.

Protein Carbonyls

The decreases in protein carbonyl concentrations were highly significant after 6 weeks (p<0.0001) and 12 weeks (p<0.0001) of supplementation (Table 1) compared to baseline. Pinus radiata bark extract reduced protein carbonyls, 51 and 42 percent after six and twelve weeks of supplementation respectively (Table 1). However no significant difference was observed between the 6 and 12 weeks of supplementation.

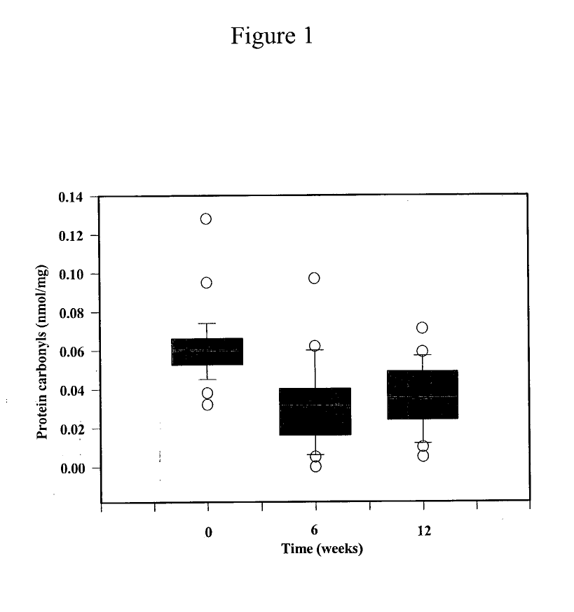

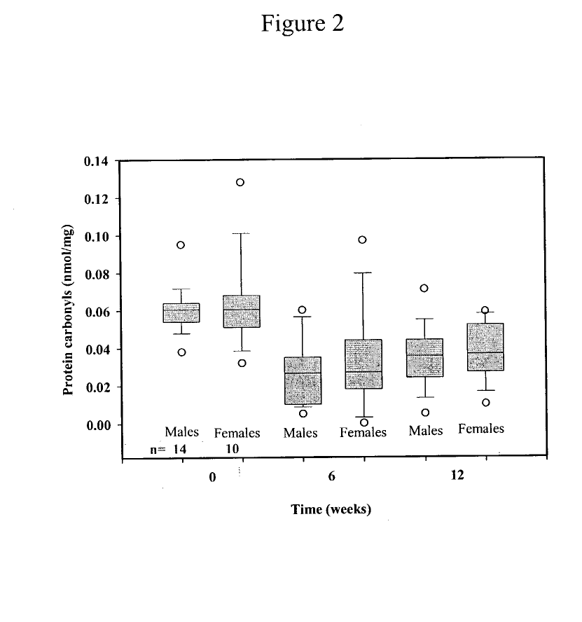

The results are also presented in FIG. 1, in which box plots show medians with interquartile ranges and error bars showing the 10% to 90% range. FIG. 2 plots the same results on a gender basis.

DNA Damage

The baseline, 6-week and 12-week samples were compared using paired t-test (Table 2). For each sample of 100 comets the distribution of tail moment for all 24 subjects had the same pattern where the distribution of tail moments can be expressed as chi-square distribution as shown by Bauer et al. (1998). Table 3 shows the average tail moment for each subject at baseline, 6 weeks and 12 weeks. DNA damage reduction as measured by the Comet assay was not significant after 6 weeks (p<0.6900) but highly significant after 12 weeks (p<0.0079) of supplementation. Pinus radiata extract reduced DNA damage by 41% after 12-weeks of supplementation.

A further test was completed where the samples used in the Comet assay above were also further analysed by measurement of 20,000 cells per patient per period using flow cytometry methodology (O'Brien et al 1997). A smaller sample was analysed (n=16) as viable samples were not available for the omitted patients. In addition, flow cytometry tests were only completed for baseline and 12-week intervals. The results in Table 4 show a marked decrease (23%) in cells exhibiting cellular DNA damage.

Total Antioxidant Activity

The ORAC results of Pinus radiata and other commercial extracts are given in Table 4, showing that Pinus radiata has a high ORAC value.

Discussion

The results indicate that both the oxidized protein marker and the DNA damage marker showed highly significant decreases over the course of the 12 week trial. However, the time period of most change was in the base-line to 6-week period for the oxidized proteins and the 6-12 week period for the DNA damage decrease.

These results are consistent with the hypothesis that antioxidants can decrease markers of oxidative damage. This has been shown previously by reduction of DNA damage in elderly people supplemented with fruit and vegetables extracts (Smith et al. 1999) and a correlation between DNA damage and low antioxidant levels in elderly (Mendoza-Nunez et al. 2001). The proathocyanidin-rich extract of Pinus radiata bark used in this study provides a highly concentrated source of antioxidants that can be incorporated into supplements and foods. This type of natural extract provides a different antioxidant activity to the majority of studies that focus on the ability of vitamin C, vitamin E and β-carotene to lower oxidative damage.

McCall and Frei (1999) recently summarized the scientific evidence for supplementation of humans with antioxidants on oxidative DNA, lipid and protein bio-markers and concluded that there was insufficient evidence to conclude that antioxidant vitamin supplementation can reduce oxidative damage in humans. However studies of cellular DNA oxidation have provided support for the protection of DNA from oxidation although some studies indicated that vitamin C supplementation could increase in DNA damage in vivo. Increasing quercetin intake in the diet did not result in increased DNA protection in vivo (Beatty et al. 2000) but the flavonoids quercetin and myricetin (Duthie et al. 1997) and the isoflavonoids genistein and equol (Sierens et al. 2001) did protect against DNA damage in vivo. Supplementation of the diet with kiwifruit (Collins et al. 2001) or tomato (Riso et al. 1999) was noted to increase the ability of lymphocytes to resist oxidative damage against DNA damage but there has been no equivalent study on the effects of polymeric proanthocyanidins.

In a clinical trial, following 5 weeks supplementation with vitamin C (400 mg/day), plasma ascorbate increased but no significant effect on protein oxidation of immunoglobulin was observed. However at 10 and 15 weeks supplementation, carbonyl levels of immunoglobulin were significantly reduced in subjects with low baseline ascorbate but not in those with normal base line ascorbate.

TABLE 1
The effects of Pinus radiata bark extract supplementation on plasma
protein carbonyl (PC) concentrations (±Std. Dev.) in older subjects
(n = 24). Using paired t-test, highly significant (p < 0.0001)
results were observed in both 6 and 12 weeks of supplementation in
comparison with baseline.

| Time | Base line | After 6 weeks | After 12 weeks |
|---|---|---|---|
| Mean of PC | 0.0621 ± 0.0187 | 0.0305 ± 0.0226 | 0.0363 ± 0.0164 |
| (nmol/mg) | | | |
| Paired t- test | | P < 0.0001 | P < 0.0001 |
| % of Carbonyl | | 50.8 | 41.5 |
| Reduction | | | |

TABLE 2
DNA comet moments at base line, after 6 and 12 weeks of
supplementation of Pinus radiata bark extract in
older subjects (n = 24). Using paired t-test,
significant (p < 0.0001) results was observed in 12 weeks of
supplementation in comparison with baseline.

| Time | Base line | After 6 weeks | After 12 weeks |
|---|---|---|---|
| Mean of DNA | 3.51 | 3.22 | 2.04 |
| Comet Moments | | | |
| Paired t- test | | P < 0.6900 | P < 0.0079 |
| % of moment | | 8.3 | 41.9 |
| reduction | | | |

TABLE 3
Subject characteristics and averaged tail moment.

| | Tail | Tail | Tail | Critical F Value | | |
|---|---|---|---|---|---|---|
| Subject | Moment | Moment | Moment | (n = 100) = 3.89 | | |
| Number | Baseline | 6 Week | 12 Week | F-Value | SEX | AGE |
| 1 | 5.40 | 1.20 | 0.72 | 24.38 | M | 65 |
| 3 | 3.78 | 1.69 | 1.20 | 43.25 | F | 66 |
| 4 | 3.07 | 1.73 | 1.27 | 5.49 | F | 69 |
| 5 | 4.59 | 2.85 | 1.77 | 88.89 | M | 60 |
| 6 | 5.48 | 10.21 | 7.20 | 1.99 | M | 59 |
| 7 | 2.71 | 1.77 | 1.93 | 6.03 | F | 60 |
| 8 | 2.00 | 2.26 | 1.64 | 4.69 | F | 65 |
| 9 | 2.18 | 2.47 | 4.11 | 6.93 | F | 75 |
| 10 | 4.68 | 1.89 | 1.50 | 23.03 | M | 69 |
| 11 | 2.70 | 1.73 | 2.82 | 0.08 | M | 73 |
| 12 | 1.84 | 2.78 | 2.35 | 2.02 | F | 70 |
| 13 | 4.45 | 3.38 | 3.40 | 1.99 | M | 68 |
| 14 | 12.70 | 3.06 | 2.33 | 37.02 | F | 56 |
| 15 | 6.51 | 4.83 | 3.51 | 21.47 | M | 71 |
| 17 | 4.28 | 2.77 | 4.00 | 0.15 | M | 60 |
| 19 | 4.35 | 14:24 | 2.02 | 25.30 | F | 58 |
| 20 | 1.81 | 3.71 | I 0.48 | 14.68 | M | 68 |
| 21 | 2.58 | 1.29 | 0.69 | 15.63 | M | 60 |
| 22 | 0.84 | 0.51 | 0.26 | 46.40 | M | 59 |
| 23 | 2.35 | 3.57 | 0.35 | 32.74 | M | 58 |
| 24 | 1.19 | 0.92 | 0.51 | 51.08 | F | 70 |
| 25 | 0.73 | 1.97 | 0.96 | 1.11 | M | 57 |
| 26 | 1.82 | 4.00 | 1.76 | 32.65 | F | 62 |
| 27 | 2.08 | 2.55 | 2.24 | 0.74 | M | 58 |

| | | | | |
|---|---|---|---|---|
| Mean | 3.51 | 3.22 | 2.04 | 6.13 (n = 24) |
| STD | 2.50 | 3.01 | 1.57 | |

TABLE 4
Flow cytometry subject characteristics and averaged tail moment.

| | Tail Moment | Tail Moment |
|---|---|---|
| Subject Number | Baseline | 12 Week |
| 3 | 14.2 | 1.8 |
| 4 | 3.9 | 5.9 |
| 7 | 69.1 | 21 |
| 8 | 81.6 | 6.8 |
| 9 | 3.1 | 5.6 |
| 10 | 30.1 | 5.1 |
| 11 | 10.8 | 32.5 |
| 12 | 23.9 | 26.3 |
| 17 | 83.5 | 2.8 |
| 18 | 54.4 | 6.5 |
| 19 | 28.7 | 17 |
| 20 | 15.4 | 9.9 |
| 21 | 44.7 | 1.3 |
| 23 | 10.7 | 5.2 |
| 26 | 14.2 | 2.5 |
| 27 | 35.2 | 3.5 |
| Avg. | 32.7% | 9.6% |

Thus, the present invention provides a method of treatment of a mammal including administration of a flavonoid extract in a combination treatment including at least one of the treatments selected from the group including: reducing a redox imbalance associated with disease in a mammal; reducing oxidative stress in a mammal; preventing oxidative stress in a mammal; reducing protein oxidation in a mammal; preventing protein oxidation in a mammal; reducing DNA damage in a mammal; preventing DNA damage in a mammal; and combinations thereof.

While a particular embodiment of the Flavonoid Extract has been described herein, it will be appreciated by those skilled in the art that changes and modifications may be made thereto without departing from the invention in its broader aspects and as set forth in the following claims.

References

Ames B. N, Shigenaga M. K. Oxidants are a major contributor to aging. Ann NY Acad Sci 1992; 663: 85-96

Bauer E, Pecknagel R D, Fiedler U, Wollweber, L, Bock Claudia, and Greulich K O: The distribution of the tail moments in single cell gel electrophoresis (comet assay) obeys a chi-square (χ²) not a gaussian distribution. Mutation Research 1998; 398; 101-110.

Beatty E R, O'Reilly D O, England T G, McAnlis G T, Young I S, Halliwell B Geissler C A Sanders T A B & Wiseman H (2000) Effect of dietary quercetin on oxidative DNA damage in healthy human subjects British Journal of Nutrition, 84, 919-925

Block, G., Patterson, B., & Subar, A. (1992). Fruit, vegetable and cancer prevention: a review of the epidemiological evidence. Nutrition and Cancer, 18, 1-29.

Buss H, Chan T P, Sluis K B, Domigan N M, and Winterbourn C C: Protein carbonyl measurement by a sensitive ELISA method. Free Radical Biology and Medicine 1997; 23; 3; 361-366

Cao G, Alessio H M, and Cutler R G: Oxygen-radical absorbance capacity assay for antioxidants. Free Radical Biology & Medicine 1993; 14; 303-311.

Carney, J., Starke-Reed, P., Oliver, C., Landum,, R., Cheng, M., and Wu, J. (1991) Proc. Natl. Acad. Sci. USA 88, 3633-3636.

Carty, J. L., Beavan, R., Waller, H., Mistry, N., Cooke, M., Lunec, J., and Griffiths, H. R. The effects of vitamin C supplementation on protein oxidation in healthy volunteers. Biochemical and Biophysical Research Communications 2000; 273; 729-735.

Ciolino H P, Levine R L: Modification of proteins in endothelial cell death during oxidative stress. Free Radic Biol Med 1997; 22: 1277-1282

Collins A Dusinska M, Franklin M,Somorovska M,Petrovska H, Duthie S, Fillion L, Panayiotidis M, Raslova K & Vaughan N (1997) Comet assay in human biomonitoring studies: Reliability, validation, and applications. Environmental and Molecular Mutagenesis 30, 139-146.

Collins B H, Horska A, Hotten P M Riddoch C and Collins A R (2001) kiwifruit protects against oxidative damage in human cells and In vitro . Nutrition and Cancer 39 148-153

Duthie S J, Collins A R, Duthie C G & Dobson V L. (1997) Quercetin and myricetin protect against hydrogen peroxide induced DNA damage (strand breaks and oxidised pyrimidines) in human lymphocytes. Mutation Research 393, 223-231.

Duthie S J, Johnson W & Dobson V L (1997) The effect of dietary flavonoids on DNA damage (strand breaks and oxidised pyrimidines) and growth in human cells. Mutation Research 390, 141-151

Frei, B., Stocker, R., and Ames, B. N. (1992) Small molecule antioxidant defences in human extracellular fluids. In The Molecular Biology of Free Radical Scavenging Systems (Scandalios, J., E d.) Cold Spring Harbor Laboratory Press, NY.

Griffiths H. R., and Lunec, J. (1998) Molecular aspects of free radical damage in inflammatory autoimmune pathology. In Molecular Biology of Free Radicals in Human Diseases (Aruoma, O. I., and Halliwell, B., Eds.), pp. 327-366, OICA International.

Harman D. The aging process. Proc Natl Acad Sci USA 1981; 78: 7124-8

Hertog, M. L. G., Feskens, E. J. M., Hollman, P. H. C., Katan, M. B. & Kromhout, D. (1993). Dietary antioxidants flavonoids and the risk of coronary heart disease: the zutphen elderly study. Lancet, 342, 1007-1011.

Johnson M K & Loo G.(2000) Effects of epigallcatechin gallate and quercetin on oxidative damage to cellular DNA. Mutation Research 459 211-218.

Lodish H, Baltimore D, Berk A, Zipursky S L, Matsudaira P, Darnell J. Molecular cell biology, 3rd ed. New York: W H Freeman & Co, 1995.

Marangon, K., Devaraj, S., and Jialal I Measurement of protein carbonyls in plasma of smokers and in oxidized LDL by an ELISA. Clinical Chemistry 1999; 45; 4; 577-578.

Markham, K. R., & Porter, L. J. (1973). Extractives of Pinus radiata bark. New Zealand Journal of Science, 16, 751-761.

McCall, M. R., and Frei, B. Can antioxidant vitamins materially reduce oxidative damage in humans? Free Radical Biology and Medicine 1999; 26; 1034-1053.

Mendoza-Nunez V M,Sanchez-Rodriquez, M A, Retana-Uqalde Rvargas-Guadarrame, L A,& Altamiro-Lozano M A (2001) Total antioxidant levels, gender, and age as risk factors for DNA damage in lymphocytes of the elderly Mechanisms of Ageing and Development 122 835-847

Middleton, E., & Kandaswami, C. (1993). The impact of plant flavonoids on mammalian biology: implications for immunity, inflammation and cancer. In: J. B. Harborne, The flavonoids: advances in research since 1986, (pp. 619-652). London: Chapman and Hall.

Middleton E. (1996) Biological properties of plant flavonoids: an overview. International Journal of Pharmacognosy; 34, 344-348

Niwa Y, Ishimoto K, Kanoh T. Induction of superoxide dismutase in leukocytes by paraquat: correlation with age and possible predictor of longevity. Blood 1990; 76: 835-41

Noroozi M, Angerson W J, and Lean M E J (1998) Effects of flavonoids and vitamin C on oxidative DNA damage to human lymphocytes. Am J Clin Nutr 1998;67:1210-8.

Olive P L and Banath J P: Induction and rejoining of radiation-induced DNA single-strand breaks: “tail moment” as a function of position in the cell cycle. Mutation Resarch 1993; 294(3); 275-283.

Oliver, C., Ahn, B., Moerman, E., Goldstein, S., and Stadtman, E. (1987) J. Biol. Chem. 262; 5488-5491

Packer, L., Rimbach, G., & Virgili, F. (1999). Antioxidant activity and biologic properties of a procyanidin-rich extract from pine (Pinus maritima) bark, Pycnogenol. Free Radical Biology & Medicine, 27, 704-724.

Porter, L. J. (1974). Extractives of Pinus radiata bark. New Zealand Journal of Science, 17, 213-218.

Pryor W A & Squadrito, G. L., (1995) The chemistry of peroxynitrite: a product from the reaction of N O with O2.—. Am. J. Physiol. 268, L699

Riso P, Pinder A, Santangelo A, Porrini M (1999) Does tomato consumption effectively increase the resistance of lymphocyte DNA to oxidative damage? Am. J. Clin. Nutr 96 712-718

Santa Maria C, Machado A. Effect of development and ageing on: pulmonary NADPH-cyt C reductase, glutathione peroxidase, glutathione reductase and thioredoxin reductase activities in male and female rats. Mech ageing Dev 1987; 37: 183-95

Sierens J, Hartley J A, Campbell M J Leathem A J C & Woodside J V (2000) Effect of phytoestrogen and antioxidant supplementation on oxidative DNA damage assessed using the comet assay. Mutation Research 485 169-176

Singh N P, McCoy M T, Tice R R and Schneider E L: A simple technique for quantitation of low levels of DNA damage in individual cells. Experimental Cell Research 1988; 175; 184-191.

Smith M J, Inserra P F, Watson R R, Wise J A, & O'Neill K L (1999) Supplementation with fruit and vegetable extracts may decrease DNA damage in the peripheral lymphocytes of an elderly population. Nutrition Research 19, 1507-1518.

Sohal, R. S., Ku, H. H., Agarwal, S., Forster, M. J., and Lal, H. (1994) Mech. Ageing Dev. 74, 121-33

Stadtman E R: Metal ion catalyzed oxidation of proteins: Biochemical mechanism and biological consequences. Free Radic Biol Med 1990; 9:315-325

Stadtman, E. (1992) Science 257; 1220-1224

Tice R and Vazquez M : Protocol for the application of the ph>13 alkaline single cell gel (scg) assay to the detection of dna damage in mammalian cells. Integrated Laboratory Systems 1998.

Wood J E, Senthilmohan S T & Peskin A V (2002) Antioxidant activity of procyanidin-containing plant extracts at different pHs. Food Chemistry v77 155-161.

Winterbourn, C. C. (1995). Nutritional antioxidants: their role in disease prevention. New Zealand Medical Journal, 108, 447-448.

Winterbourn C C, and Buss H: Protein carbonyl measurement by enzyme-linked immunosorbent assay. Methods in Enzymology 1999; 300; 106-111

What is claimed:

1. The use of a flavonoid extract in a treatment of a mammal for at least one of the treatments selected from the group consisting of: reducing a redox imbalance associated with disease in a mammal; reducing oxidative stress in a mammal; preventing oxidative stress in a mammal; reducing protein oxidation in a mammal; preventing protein oxidation in a mammal; reducing DNA damage in a mammal; preventing DNA damage in a mammal; and combinations thereof.

2. The use of a flavonoid extract as claimed in claim 1 wherein the extract is an anti-oxidant extract.

3. The use of a flavonoid extract as claimed in claim 1 wherein the extract is a proanthocyanidin-rich extract.

4. The use of a flavonoid extract as claimed in claim 3 wherein the proanthocyanidin-rich extract is a pine bark extract.

5. The use of a flavonoid extract as claimed in claim 4 wherein the pine bark extract is from Pinus radiata .

6. The use of a flavonoid extract as claimed in claim 1 wherein the formulation includes at least one additional anti-oxidant.

7. The use of a flavonoid extract as claimed in claim 6 wherein the anti-oxidant is vitamin C, and the ratio of extract to vitamin C is 2:1 by weight.

8. The use of a flavonoid extract as claimed in claim 1 wherein the extract is included in a dietary supplement formulation.

9. The use of a flavonoid extract as claimed in claim 1 wherein the mammal is a human being:

10. The use of a flavonoid extract as claimed in claim 9 wherein the daily dosage of said extract for a human is from 5 mg per day to 1500 mg per day.

11. The use of a flavonoid extract as claimed in claim 9 wherein the daily dosage of said extract for a human is 480 mg per day.

12. The use of a flavonoid extract as claimed in claim 1 wherein the extract is taken orally.

13. The use of a flavonoid extract as claimed in claim 1 wherein the extract is administered in a method selected from the group consisting of: a tablet; a capsule; a suppository; an injection; a suspension; a drink; a tonic; a syrup; a powder; an ingredient in solid foods; an ingredient in liquid foods; a topical application; and combinations thereof.

14. A method of treatment of a mammal including administration of a flavonoid extract in a combination treatment including at least one of the treatments selected from the group consisting of: reducing a redox imbalance associated with disease in a mammal; reducing or prevent oxidative stress in a mammal; reducing or preventing protein oxidation in a mammal; reducing or preventing DNA damage in a mammal; and combinations thereof.

15. The use of a flavonoid extract as claimed in claim 14 wherein the extract is an anti-oxidant extract.

16. The use of a flavonoid extract as claimed in claim 14 wherein the extract is a proanthocyanidin-rich extract.

17. The use of a flavonoid extract as claimed in claim 16 wherein the proanthocyanidin-rich extract is a pine bark extract.

18. The use of a flavonoid extract as claimed in claim 17 wherein the pine bark extract is from Pinus radiata.

19. The use of a flavonoid extract as claimed in claim 14 wherein the formulation includes at least one additional anti-oxidant.

20. The use of a flavonoid extract as claimed in claim 19 wherein the anti-oxidant is vitamin C, and the ratio of extract to vitamin C is 2:1 by weight.

21. The use of a flavonoid extract as claimed in claim 14 wherein the extract is included in a dietary supplement formulation.

22. The use of a flavonoid extract as claimed in claim 14 wherein the mammal is a human being.

23. The use of a flavonoid extract as claimed in claim 22 wherein the daily dosage of said extract for a human is from 5 mg per day to 1500 mg per day.

24. The use of a flavonoid extract as claimed in claim 22 wherein the daily dosage of said extract for a human is 480 mg per day.

25. The use of a flavonoid extract as claimed in claim 14 wherein the extract is taken orally.

26. The use of a flavonoid extract as claimed in claim 14 wherein the extract is administered in a method selected from the group consisting of: a tablet; a capsule; a suppository; an injection; a suspension; a drink; a tonic; a syrup; a powder; an ingredient in solid foods; an ingredient in liquid foods; a topical application; and combinations thereof.
